(19) United States
(12) Patent Application Publication
(10) Pub. No.: US 2024/0050095 A1
(43) Pub. Date: Feb. 15, 2024

(54) DEVICE FOR ATTACHING AN IMPLANT

(71) Applicant: MEDIZINISCHE UNIVERSITÄT WIEN, Wien (AT)

(72) Inventors: Ewald UNGER, Wien (AT); Marek EHRLICH, Wien (AT); Emilio OSORIO, Wien (AT)

(73) Assignee: MEDIZINISCHE UNIVERSITÄT WIEN, Wien (AT)

(21) Appl. No.: 18/266,856

(22) PCT Filed: Dec. 16, 2021
(86) PCT No.: PCT/IB2021/061846

§ 371 Date: Jun. 13, 2023

(30) Foreign Application Priority Data
Dec. 16, 2020 (AT) A 275/2020

(51) Int. Cl. A61B 17/115; A61B 17/064

(52) U.S. Cl. CPC A61B 17/115; A61B 17/0644; A61B 2017/1107

(57) ABSTRACT

A device for attaching a primary implant as a fixation point for a vascular prosthesis on a vessel wall of a blood vessel, the device at least including a carrier element, which is designed to drive a plurality of penetration elements, for penetrating the vessel wall, radially outwards in relation to a longitudinal axis of the carrier element. In the device, a limiting element is formed on the carrier element as a radial stop for the penetration elements.

### CROSS-REFERENCE TO RELATED APPLICATIONS

The present application is a national phase application of PCT Application No. PCT/IB2021/061846, filed Dec. 16, 2021, entitled “DEVICE FOR ATTACHING AN IMPLANT”, which claims the benefit of Austrian Patent Application No. A 275/2020, filed Dec. 16, 2020, each of which is incorporated by reference in its entirety.

### BACKGROUND OF THE INVENTION

### 1. Field of the Invention

The present invention relates to a device for attaching a primary implant as a fixation site for a vascular prosthesis to a vessel wall of a blood vessel, the device at least comprising a carrier member configured to drive a plurality of penetrating members radially outwardly with respect to a longitudinal axis of the carrier member for penetrating the vessel wall.

### 2. Description of the Related Art

Dissection of the ascending aorta (type A dissection) is a life-threatening condition that requires immediate surgery. In acute type A dissection, the primary goal of surgical therapy is oriented toward restoring circulation and preventing or reversing life-threatening complications, such as aortic rupture, pericardial tamponade, and myocardial ischemia. The further goal of surgical therapy is, if possible, resection of the tear of the tunica intima (the entry port into the false lumen) in the proximal aorta to ensure perfusion of the true lumen and thus of the organs.

Classical surgical therapy for acute type A dissection involves replacement of the ascending aorta with a vascular prosthesis in the form of a tubular prosthesis, with distal anastomosis performed using an “open” technique and hypothermic circulatory arrest at approximately 25° C. core body temperature.

The surgical approach is through a median sternotomy. With the use of the heart-lung machine and cooling of the patient, circulatory arrest is started in head-down position and the ascending aorta can be opened. The true lumen is identified and the ascending aorta is resected to its distal portion so that the distal ascending aorta can be viewed like an open tube.

Now, in the currently common surgical procedure, the shattered or delaminated parts of the aortic wall (tunica intima, tunica media and tunica adventitia) are sutured together in a so-called “sandwich” technique. This is done by placing a primary implant, for example a felt strip, i.e. a strip of textile material, circularly or radially inside the aorta and, if necessary, a secondary implant, for example a felt strip, circularly or radially on the outside of the aorta. A continuous meandering suture is then performed manually using polypropylene suture, which sutures these two felt strips and the aortic wall in between to each other (sandwich).

This step of the surgery takes about 25 minutes, but may well be ten minutes faster or slower depending on surgical experience. Following this, in a second step, a tubular prosthesis is now continuously sutured to this sandwich stump, the fixation site, by hand using a polypropylene suture end-to-end.

After the appropriate deflation maneuver, an aortic clamp can now be placed on the now sewn-on prosthesis and the circulatory arrest is considered to be lifted. The most critical phase of the surgery is thus usually mastered. A sandwich of the native aorta is then also performed supracoronary at the proximal end near the heart using the same technique described above. The proximal end of the prosthesis is then sutured to this sandwich (fixation site) end-to-end as described above.

It is obvious that in such a profound operation, where the normal blood circulation is interrupted and the body and brain are severely cooled, the time factor is of immense importance in order to optimize the chances of survival as well as the recovery of the patient. In particular, the procedure described above for treating the dissection by applying the sandwich suture depends to a large extent on the individual circumstances of the patient, the extent of the dissection of the vessel wall and, last but not least, the skill of the surgeon.

Devices have therefore already been developed to simplify and speed up the placement of the primary implant and, if necessary, the secondary implant, in particular by replacing the time-consuming step of setting the polypropylene suture in the form of a continuous, meandering suture with machine insertion of penetrating members such as staples. For this purpose, devices have been created to push the penetrating members outward from a carrier member and thereby drive them through the primary implant as well as the vessel wall and, if necessary, through the secondary implant.

A disadvantage of these devices, however, is that penetration of the primary implant as well as the vessel wall can sometimes result in high strain loads for the already damaged blood vessel.

### SUMMARY OF THE INVENTION

The invention is therefore based on the task of improving the largely mechanized attachment of primary implants, which in the context of the present invention are to be understood as support structures for the vessel wall, such as stents and/or felt strips to a vessel wall, in particular to the aortic wall in humans, by creating a corresponding device in such a way that high strain loads are avoided.

To solve this problem, a device of the type described above has a restraining member formed on the carrier member as a radial stop for the penetrating members. The fact that a restraining member is provided as a radial stop, i.e. as a boundary surrounding the blood vessel, means that stretching stresses on the blood vessel are largely avoided and the penetrating members can be placed particularly quickly and without the risk of tearing the blood vessel.

In order to adapt the device to different anatomical conditions, the device according to the invention is preferably further designed in that the restraining member has an adjustable diameter.

The restraining member may be provided as an independent component of the device according to the invention. However, it may also be provided that the restraining member is formed integrally with the carrier member, as corresponds to a preferred embodiment of the present invention.

Preferably, the present invention is further configured in that the restraining member is formed as a ring surrounding the carrier member. This represents a particularly simple variant of the restraining member of the device according to the invention, with which, however, good outward support of the vessel wall can already be achieved.

According to an alternative preferred embodiment of the present invention, the restraining member is formed by a plurality of plates fixed to the carrier member, preferably at a radially adjustable distance from the longitudinal axis. The plates are formed here to surround the vessel outwardly to support the vessel wall, the plates preferably being arranged along the circumference of an imaginary circle and consequently surrounding the blood vessel in a flower-like manner. This allows, in order to adjust the diameter of the restraining member formed by the plates, to bend the plates accordingly before inserting the carrier member into the lumen of the vessel. However, it is also conceivable in a preferred manner that the plates can be adjusted with regard to their distance from the carrier axis by means of a corresponding mechanism.

According to a preferred embodiment of the present invention, the primary implant is arranged radially outside the carrier member as a part of the device releasably fixed to the device, wherein the primary implant is penetrable by the penetrating members and is preferably radially expandable. This means that the device according to the invention is already equipped with the primary implant, so that the primary implant can be inserted into the lumen of the vessel together with the insertion of the carrier member of the device according to the invention and subsequently can be immediately fixed to the vessel wall by driving the penetrating members into the primary implant and the vessel wall.

As a variant of the primary implant, according to a preferred embodiment of the present invention, it may be provided that the primary implant is formed as a tube of textile material. The primary implant is comparable to the aforementioned felt strip and is expanded by the expandable carrier member to come into contact with the vessel wall in order to attend to the dissection in this way.

According to a preferred embodiment of the present invention, in order to ensure the expandability of the primary implant, it may be provided that the tube of textile material has a cut-through, preferably toothed, in its circumference. The cutting allows the primary implant to expand from a small diameter for easy insertion into the lumen of the blood vessel as the support structure expands and thus comes into contact with the vessel wall.

According to a further preferred variant, the present invention is characterized with respect to the primary implant in that the primary implant is designed as a metallic tube with lattice walls. In this case, the primary implant of the device of the invention is substantially similar to a conventional metallic stent that is brought to bear against delaminated vessel walls to attend to them.

According to a further preferred variant, the present invention is characterized with respect to the primary implant in that the primary implant is designed as a ring with deformable expansion regions. The deformable expansion regions in this case can be regions with coiled connecting webs between circumferential sections of the ring, which are pulled apart when the carrier member and thus, if applicable, the primary implant, expands.

To define the aforementioned primary implants, the carrier member has a radially expandable common support structure for the plurality of penetrating members, in accordance with a preferred embodiment of the present invention. Thus, the penetrating members are located and retained on or within the carrier member and can be driven outwardly by the radially expandable support structure to penetrate the primary implant and, subsequently, the vessel wall, thereby attaching the primary implant to the vessel wall.

Alternative primary implants are described below that already have the penetrating members and therefore do not need to be attached to the vessel wall according to the method of attachment just mentioned. This ensures a particularly rapid attachment of the primary implant with the device according to the invention by suturing to the vessel wall.

According to a preferred embodiment of the present invention, the primary implant in this context is formed as a tube of textile material and has penetrating members as radially outwardly extending needle-like projections, the tube of textile material having a, preferably toothed, separation in its circumference. The primary implant is again comparable to the aforementioned felt strip and is expanded by the expandable carrier member to come into contact with the vessel wall in order to supply the dissection in this way. However, the primary implant itself has the penetrating members, so they do not need to be located on or in the carrier member.

Alternatively, with regard to the primary implant, the device according to the invention can preferably be further configured in that the primary implant is designed as a metallic tube with lattice walls and has penetrating members as radially outwardly directed, needle-like projections which are fixed to the lattice walls, preferably formed integrally therewith. In this case, the primary implant of the device of the invention is again substantially similar to a metallic stent that is brought to bear against the delaminated vessel wall to attend to it. However, in this case, the primary implant itself has the penetrating members, so they do not need to be located on or in the carrier member.

According to another alternative and preferred embodiment of the present invention, the primary implant is formed as a ring with deformable expansion regions and has penetrating members as radially outwardly extending needle-like projections that are fixed to the ring, preferably formed integrally therewith. The deformable expansion regions in this case can be regions with coiled connecting webs between circumferential sections of the ring, which are pulled apart as the carrier member expands. Also in this case, the primary implant itself has the penetrating members, so they do not need to be located on or in the carrier member.

The primary implants described so far can be supplemented by further implants of the device according to the invention. For this purpose, according to a preferred embodiment of the present invention, it may be provided that a secondary implant in the form of a tube of textile material is arranged radially outside the penetrating members. The secondary implant in this case, regardless of the type of primary implant described above, is similar to the felt strip mentioned at the beginning and is also penetrated by the penetrating members to secure the ensemble of primary and secondary implant to the vessel wall internally and externally.

With regard to the carrier member, the device according to the invention is characterized according to a preferred embodiment in that the carrier member comprises a central push rod, to one end of which a plurality of foldable stirrups are hinged as a support structure, each of which is connected to an actuating rod, the stirrups being radially expandable by a relative axial displacement of the push rod with respect to an actuating rod of a stirrup. The stirrups are thus more or less applied to the push rod in one end position of the latter and straighten in a parallelogram-like manner when the push rod is displaced relative to the actuating rods. This provides a simple mechanism for expansion of the primary implant with the penetrating members, which can be easily operated by the surgeon and which allows a dosable contact (ie. an adjustable pressing) force of the primary implant, the penetrating members and, if necessary, the secondary implant to be applied against the vessel wall and subsequently against the restraining member. In this case, the push rod and the actuating rods can preferably be designed as flexible rods in order to make it easier to insert the carrier member according to the invention in situ at the desired location on the blood vessel. In this case, at least the push rod is designed as a Bowden cable.

In principle, two foldable stirrups may already be sufficient to tighten the vessel wall or aortic wall and to attach the primary implant and, if necessary, the secondary implant to the vessel wall. However, it is preferred that the stirrups are arranged in a star shape around the push rod in the radial direction. This means that more than two, in particular twelve to sixteen foldable stirrups are provided to circularly span the vessel wall and attach the primary implant to the vessel wall.

When more than two foldable stirrups are provided, the actuating rods of the stirrups may form a cage surrounding the push rod in which the push rod is guided, in accordance with a preferred embodiment of the present invention. In this case, the push rods are guided along all sides of the push rod and are joined at the proximal end, i.e. at the end of the carrier member that is spaced from the stirrups, for example with a circumferential ring.

Typically, in the case of simple pins, the penetrating members are either glued to the outside of the vessel or to a secondary implant radially outside the primary implant and, if applicable, to the secondary implant located radially outside the vessel wall, or, in the case of staples, they are bent over on the outside of the vessel to positively attach the penetrating members and the primary implant and, if applicable, the secondary implant to the vessel wall. Preferably, the penetrating members are made of a so-called shape-memory material, which, when heated, is converted into a bent conformation. In order to bring about the heating, according to a preferred embodiment of the present invention, the device may comprise means for heating the stirrups at least in a partial region, preferably in the form of a resistance heater. With this preferred embodiment of the invention, the penetrating members can be heated after penetrating the primary implant and the vessel wall, thereby bringing them into their recontoured conformation. In this case, it is not necessary to press the penetrating members so tightly against the restraining member that cold deformation of the penetrating members occurs, so that the fixation of the primary implant and, if necessary, the secondary implant to the vessel wall is carried out particularly gently.

According to an alternative embodiment of the present invention, the carrier member has a base body in which a threaded rod having two threaded sections with threads running in opposite directions is mounted, wherein a screw mounted in a rotationally fixed manner relative to the base body is arranged on each of the threaded sections and expansion elements, preferably made of metal in the form of sheets or wires or of plastic in the form of plates or wires, are fixed as a support structure to the screws and connecting the screws. This represents a type of expansion of the carrier member in which two counter-rotating spindle drives are formed on a single threaded rod to move the screws of the spindle relative to each other so that, as a result, the expansion elements are bent as the screws converge on the threaded rod and are thereby driven radially outward. In this case, the threaded rod can preferably be designed as a flexible rod in order to make it easier to insert the carrier member according to the invention in situ at the desired location on the blood vessel.

According to a further alternative embodiment of the present invention, the carrier member has a guide rod for a first bearing for expansion elements, which is guided displaceably along the guide rod and can be displaced, preferably by means of a cable pull, against a second bearing for expansion elements, which is not displaceably mounted on the guide rod, the expansion elements, preferably made of metal in the form of sheets or wires or of plastic in the form of plates or wires, being fixed as a support structure to the bearings and connecting the bearings. In this case, the guide rod can preferably be designed as a flexible rod in order to make it easier to insert the carrier member according to the invention in situ at the desired location on the blood vessel.

In principle, two expansion elements may already be sufficient to tighten the vessel wall or aortic wall and to attach the primary implant and, if necessary, the secondary implant to the vessel wall. However, it is preferred that the expansion elements are arranged in a star shape in the radial direction around the threaded rod or guide rod. This means that more than two, in particular four to eight, especially six expansion elements are provided to circularly expand the vessel wall and attach the primary implant to the vessel wall.

In order to apply the contact pressure to the penetrating members in a controlled manner when the carrier member expands, the invention is preferably further embodied in that the expansion elements have raised supports for the penetrating members which protrude through the base body during expansion.

The present invention is further preferably characterized in that a plurality of spring-loaded blocks, preferably metallic blocks, are secured to the guide rod and are releasable to abut the supports. With this preferred embodiment of the present invention, it is possible to bring the penetrating members into their final position with a firm stroke after penetration of the primary implant, the vessel wall and, if necessary, the second implant, whereby they are reliably bent over at the restraining member and thereby firmly anchored.

An alternative and preferred embodiment of the present invention is characterized in that the carrier member comprises a housing and, in the housing, radially outwardly directed guides for the penetrating members along which the penetrating members can be driven radially outwardly. In this case, the penetrating members do not rest on support elements, but are driven outward from the interior of a housing of the carrier member and subsequently through the primary implant, the vessel wall and, if applicable, the secondary implant, whereupon they are bent over at the restraining member and thereby anchored. In this regard, according to a preferred embodiment, the housing may be connected to a handle for actuating the device according to the invention by a flexible connecting portion.

In connection with this just disclosed embodiment of the present invention, it is preferably provided that the carrier member has a radially expandable common support structure for the plurality of penetrating members, the carrier member having a central push rod cooperating with a plurality of radially displaceable supports for the primary implant, preferably with the interposition of a link guide in each of the plurality of supports, the supports being drivable radially outwardly by an axial displacement of the push rod relative to the supports. This means that a support structure consisting of a plurality of supports is provided for the primary implant, the supports being mounted in the housing so as to be axially fixed in shear and radially displaceable. Displacement of the push rod within the housing and axially relative to the supports causes the supports to be driven radially outward, bringing the primary implant, such as a tube of textile material or a metallic tube with lattice walls or a ring with deformable expansion regions as described above, into abutment against the vessel wall from the inside. Subsequently, the penetrating members can be driven from the interior of the housing of the carrier member to the outside and subsequently through the primary implant, the vessel wall and, if applicable, the secondary implant, whereupon they are bent over at the restraining member and thereby anchored.

Preferably, the radially outwardly directed guides for the penetrating members are formed in at least two first disks which are coaxial with respect to the longitudinal axis of the carrier member and spaced apart from each other and have the guides as radially extending recesses. The guides for the penetrating members are thus formed, as it were, as recesses, such as slots or channels, running radially with respect to the longitudinal axis of the device according to the invention, in typically circular disks, so that the recesses run outwards from the longitudinal axis in a star shape. In this case, one of the recesses extending in a star shape in one of the two disks and one of the recesses extending in a star shape in the other of the two disks each form a pair of recesses which are spaced apart from one another with respect to the longitudinal axis and are opposite one another in this way, in each of which one of the penetrating members is guided and driven outwards. The arrangement of the guides in disks spaced apart from one another with respect to the longitudinal axis, the disks preferably being made of plastic, makes it possible to arrange a large number of the recesses or slots running outwards in a star shape as guides close together in the circumferential direction about the longitudinal axis in a device that is still reasonably easy to manufacture.

According to a preferred embodiment of the present invention, the penetrating members in the guides can be driven radially outward on supports for the penetrating members. In this embodiment, the penetrating members rest on supports. This makes it possible to drive even different types of penetrating members reliably and guided to the outside.

Viewed in a sectional plane extending normal to their radial extent, the guides may each have a second guide region which is widened in the circumferential direction of the disk compared to a first guide region adjoining the second guide region in the axial direction, as corresponds to a preferred embodiment of the present invention. This allows the penetrating members to be guided precisely in the narrower first guide regions and to provide sufficient space in the wider second guide regions for the mechanism used to drive the penetrating members outward.

Further, the provision of guide regions of different sizes enables better guidance of the supports for the penetrating members when the supports with thickenings engage the second guide regions, in accordance with a preferred embodiment of the present invention.

In order to create the expanded second guide regions in the simplest possible manner, the invention is preferably further configured for this purpose in that the second guide regions are formed in respective second disks adjacent to the first disks, which disks have recesses extending radially to form the second guide regions. The second disks are thus quite similar in design to the first disks and are generally only modified in that the radially extending recesses have a greater width in the circumferential direction. Two disk packs spaced along the longitudinal axis are formed in this preferred embodiment of the present invention, forming the guides for the penetrating members and, where applicable, for the supports.

According to a preferred embodiment of the present invention, the mechanism for driving the penetrating members or the supports is realized in that the disks are mounted on a common shaft which has axial recesses or bores and guide rods received therein and spiral springs held on the guide rods, wherein the spiral springs can each be at least partially pushed off the guide rods and into a guide element by a gripping element. The shaft can consist of several axial parts and be hollow in order to pass actuating rods for distally located gripping elements. The mechanism thus provides that for each guide a separate spiral spring is accommodated on a corresponding guide rod. Due to the action of the gripping elements, the springs can be at least partially pushed off the guide rods and into the guides by a corresponding shaping of the device according to the invention. When the spiral springs are pushed off the guide rods, the springs relax and the penetrating members are driven outward by the corresponding increase in length, if necessary with the interposition of the supports.

Preferably, the gripping elements of all spiral elements can be actuated together so that the penetrating members are simultaneously driven outward around the circumference of the device according to the invention.

The spiral springs are insertable into the second guide regions, as is in accordance with a preferred embodiment of the present invention. The second guide regions provide sufficient space for the springs while reliable guidance of the penetrating members is accomplished in the first guide regions, as indicated above.

### BRIEF DESCRIPTION OF THE DRAWINGS

The invention is explained in more detail below with reference to an example of embodiments shown in the drawings:

FIG. 1 shows a schematic representation of a carrier member of the device according to the invention with a support structure in an expanded state,

FIG. 2 shows a carrier member with a semi-collapsed support structure,

FIG. 3 shows a sectional view of an alternative carrier member with a primary implant in an unexpanded state,

FIG. 4 shows the carrier member according to FIG. 3 in an expanded state,

FIG. 5 shows a perspective view of a detail of the carrier member according to FIGS. 3 and 4,

FIG. 6 shows a perspective view of a variant of a primary implant of a device according to the invention,

FIG. 7 a preferred variant of a device according to the invention with a housing and in the housing radially outwardly directed guide elements for the penetrating members, with non-extended penetrating members,

FIG. 8 the device according to FIG. 7 with outwardly driven penetrating members,

FIG. 9 a perspective sectional view of the device according to FIGS. 7 and 8,

FIG. 10 a representation of a support structure for a primary implant in a device of the type according to FIGS. 7 to 8,

FIG. 11 a sectional view of the support structure of FIG. 10 in an unexpanded state,

FIG. 12 a sectional view of the support structure of FIGS. 10 and 11 in an expanded state,

FIG. 13 a detail of a variant of the device according to FIGS. 7, 8 and 9 with unexpanded penetrating members,

FIG. 14 the device according to FIG. 10 with outwardly driven penetrating members,

FIG. 15 a variant of the device according to FIG. 3,

FIG. 16 a perspective view of a preferred variant of the present invention with guides for the penetrating members formed in disks,

FIG. 17 a view according to FIG. 16 partially in section,

FIG. 18 the view of FIG. 17 with retracted spiral springs,

FIG. 19 a view of FIG. 17 with partially extended spiral springs,

FIG. 20 a sectional view of the device according to FIGS. 16 to 19 with retracted spiral springs, and

FIG. 21 a sectional view of the device according to FIGS. 16 to 19 with extended spiral springs.

### DETAILED DESCRIPTION

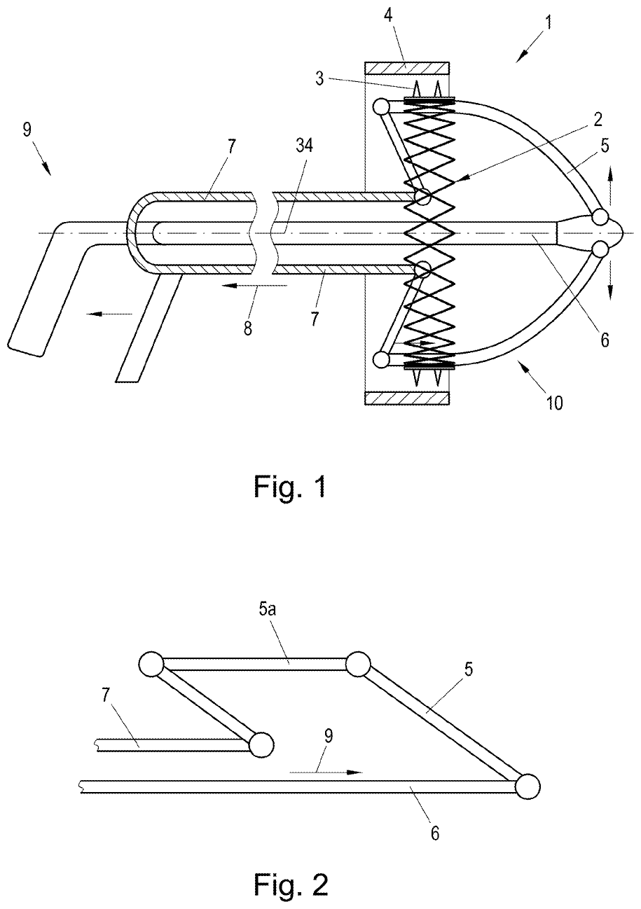

In FIG. 1, the device according to the invention is designated by the reference numeral 1. The device 1 consists of a primary implant 2 with penetrating members 3, in the example of FIG. 1 in the form of needle-like projections or hooks. Reference numeral 4 denotes a restraining member against which the primary implant can be pressed in expansion and which forms a radial stop for the penetrating members. With respect to the support structure 10, the device 1 has a plurality of foldable stirrups 5 hingedly fixed to a push rod 6. The stirrups 5 are each connected to an actuating rod 7, whereby the stirrups 5 are radially expandable by a relative axial displacement of an actuating rod 7 to the push rod 6 in the direction of the arrow 8. A plurality of actuating rods 7 are combined at the proximal end 9 of the device by a ring to form a cage in which the push rod 6 is guided.

In FIG. 2 and the following figures, identical or corresponding parts are marked with the same reference numerals. It can be seen that when the actuating rod 7 is moved relative to the push rod 6 in the direction of the arrow 9, the stirrups 5 collapse or fold together. The partial region 5a of the stirrups 5 can be heated, for example by a resistance heater, in order to be able to deform a penetrating member 3 made of shape memory material, as described above.

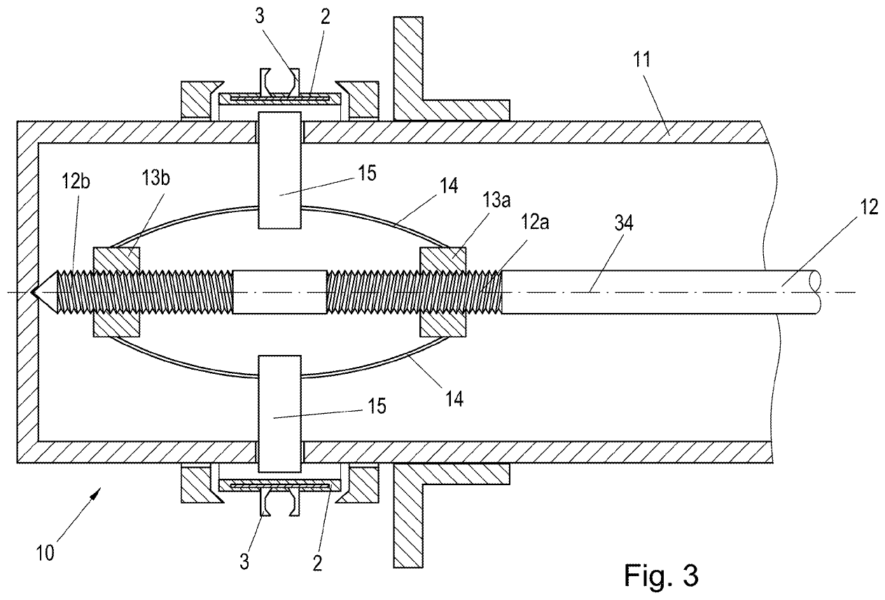

FIG. 3 shows an alternative type of support structure 10 which has a base body 11 in which a threaded rod 12 having two threaded sections 12a and 12b with threads running in opposite directions is mounted, a screw 13a and 13b which are mounted in a rotationally fixed manner relative to the base body 11 being arranged on each of the threaded sections 12a and 12b, and expansion elements 14 being fixed to the screws 13a and 13b and connecting the screws 13a and 13b. The expansion elements 14 further have raised supports 15 to apply the contact force to the penetrating members 3 in a controlled manner when the support element 10 expands. Preferably, the supports 15 may include resistance heating to allow appropriate deformation of any penetrating members made of shape memory material, as described above.

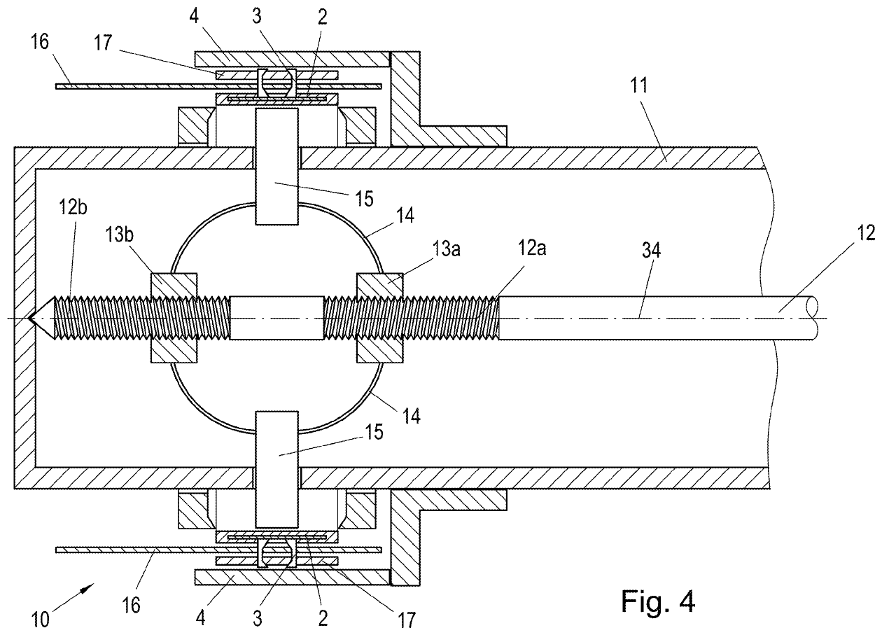

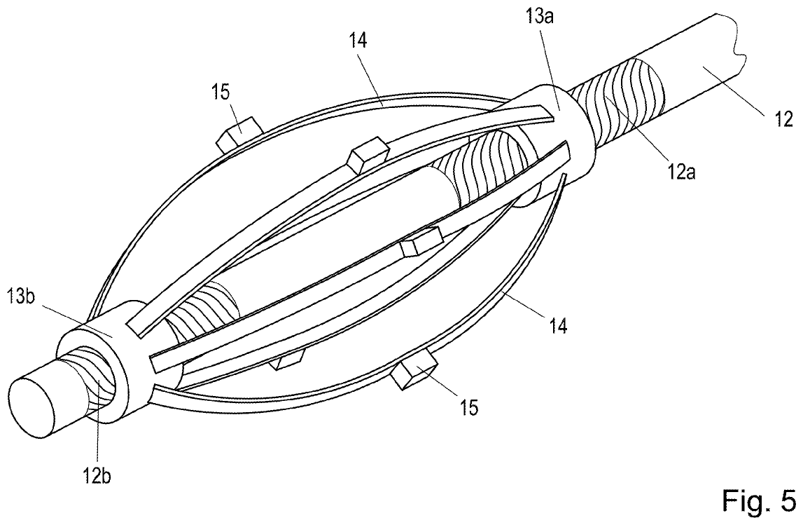

In FIG. 4, it can be seen that when the threaded rod 12 is rotated, the screws 13a and 13b on the threaded sections 12a and 12b can be moved toward each other, forcing the expansion elements 14 outward. This drives the primary implant 2 with its penetrating members 3 outward and the penetrating members 3 penetrate the vessel wall 16. Upon contact with the restraining member 4, the penetrating members 3 are bent over and thus secured. Reference numeral 17 denotes an outer, secondary implant, for example made of a nonwoven or tissue material, preferably tubular, like the primary implant 2.

In FIG. 5, the support structure 10 is shown without the base body 11 and the same parts are provided with the same reference numerals.

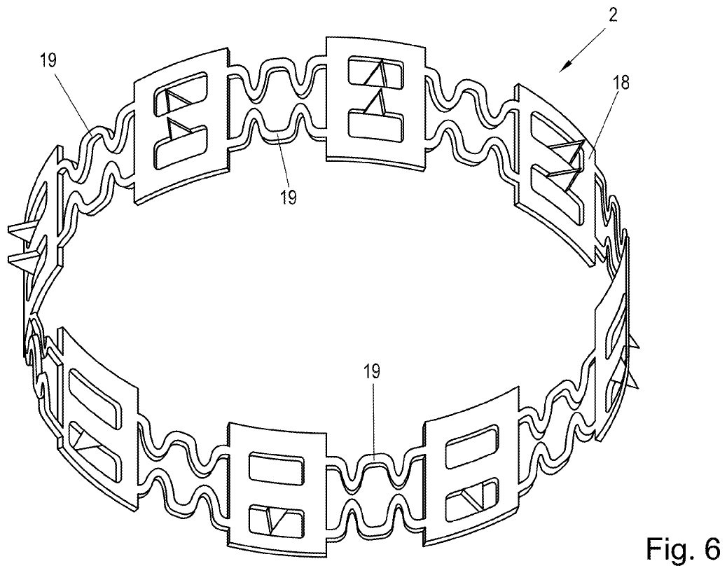

The primary implant 2 shown in FIG. 6 is not designed as a tubular stent with lattice walls, but as a ring 18 with deformable expansion regions 19. The ring 18 has penetrating members 3 as radially outwardly extending, needle-like projections which are formed integrally with the ring 18. In this example, the penetrating members 3 are stamped from the material of the ring and bent out of the outer surface of the ring 18.

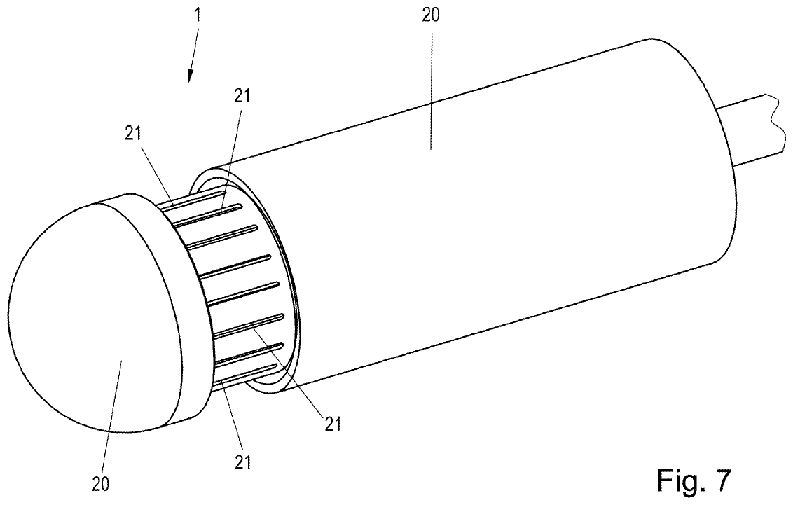

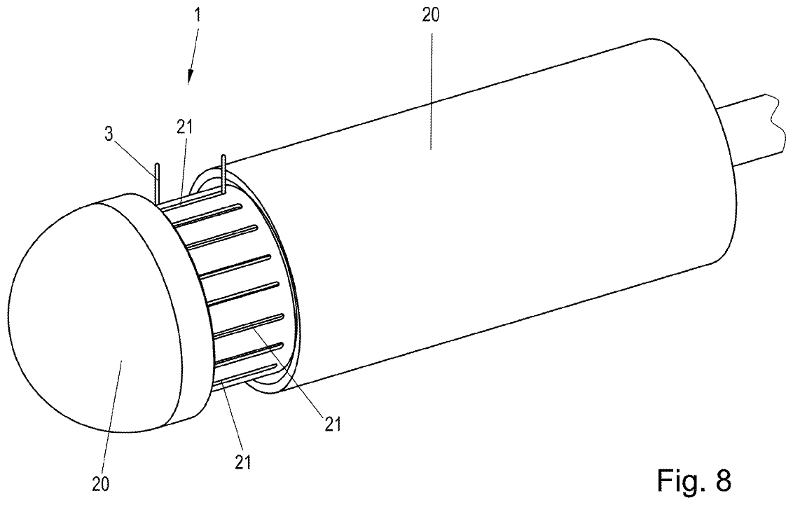

The variant of a device according to the invention in FIGS. 7 and 8 has a housing 20 in which the carrier member is arranged to provide the plurality of penetrating members for penetrating the vessel wall. Penetrating members 3 can be driven outward through slots 21 when the device 1 is actuated. FIGS. 7 and 8 do not show the restraining member according to the invention.

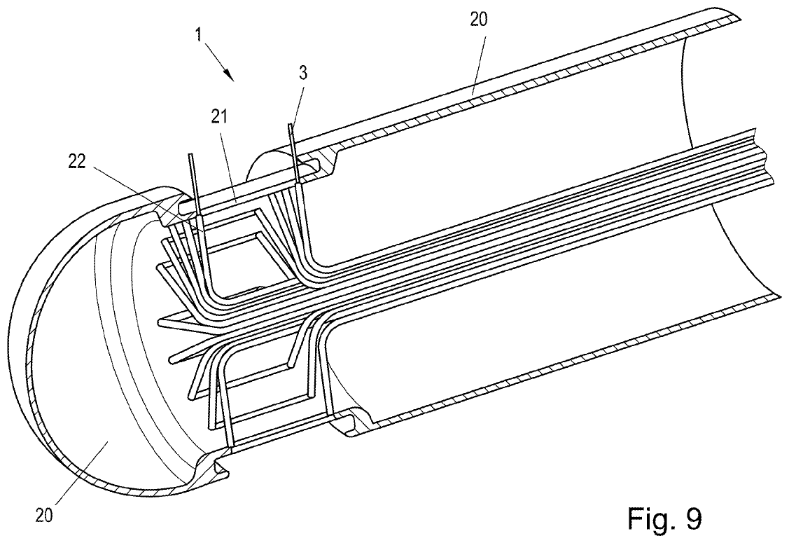

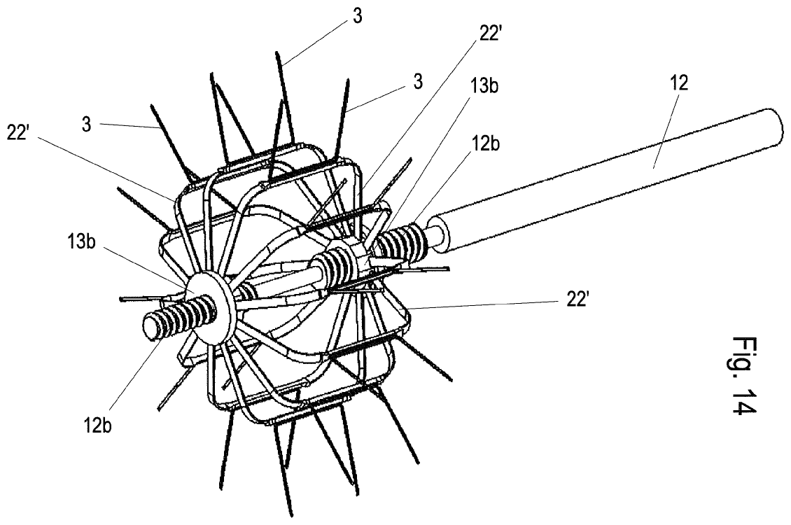

In FIG. 9, it can be seen that radially outwardly directed guide elements 22 for the penetrating members 3 are arranged in the housing 20, along which the penetrating members 3 can be driven radially outwardly. A mechanism not shown in more detail is provided for this purpose, which is illustrated by way of example in FIGS. 13 and 14.

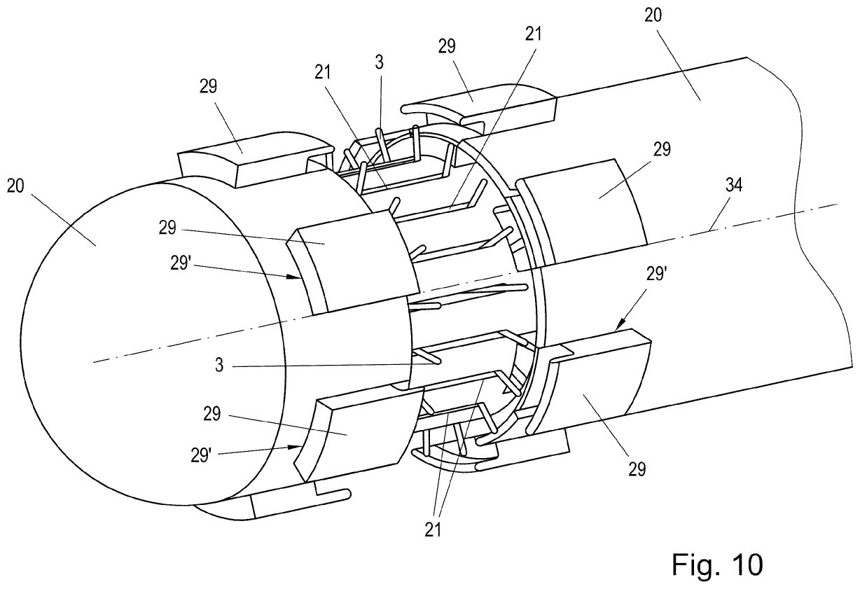

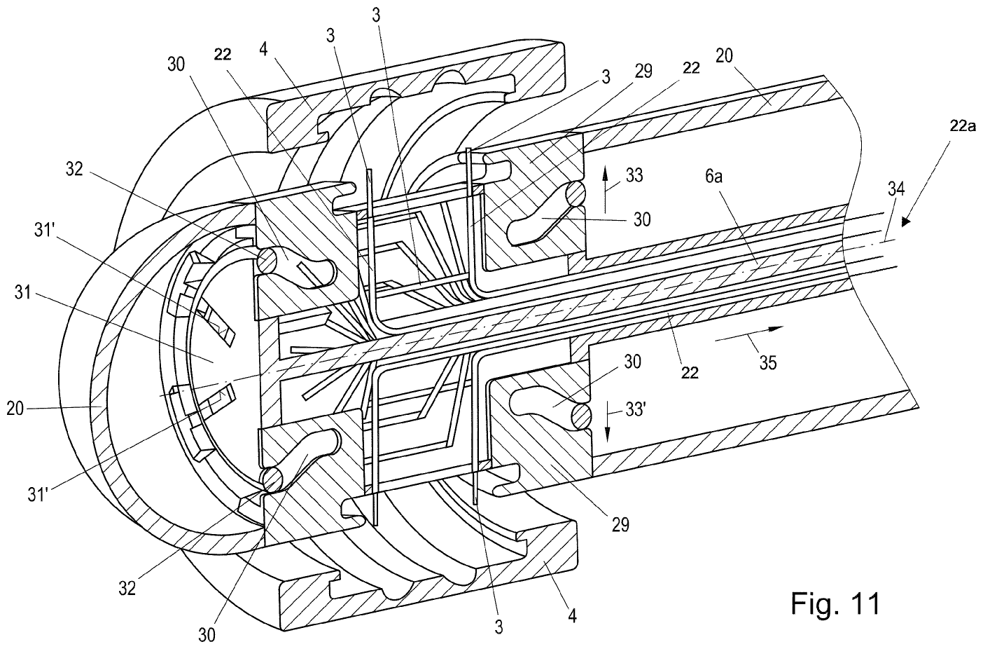

FIG. 10 shows supports 29 for a primary implant 2, the supports being radially displaceable or moveable in corresponding recesses 29′ in the housing 20. However, the supports 29 are mounted in the recesses 29′ in a shear-resistant manner in the axial direction, i.e. in the direction of the axis 34 of the carrier member in the housing 20. A primary implant can be placed on the supports 29, so that when the carrier member expands, the primary implant comes into contact with a vessel wall by displacement of the supports 29 to be subsequently secured by driving the penetrating members 3 outward. In FIG. 10, the supports 29 are shown in an expanded state.

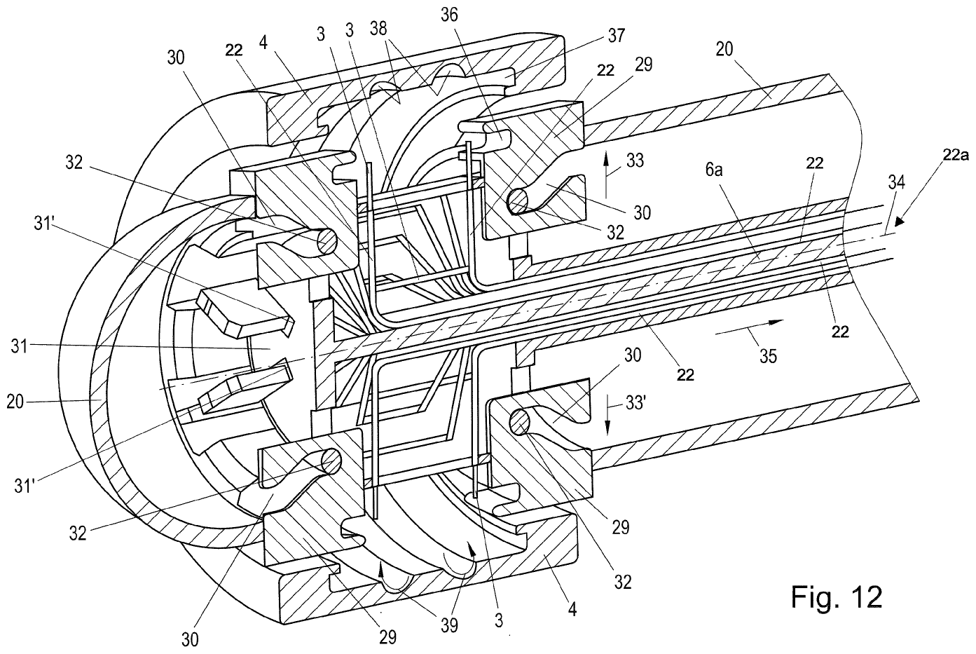

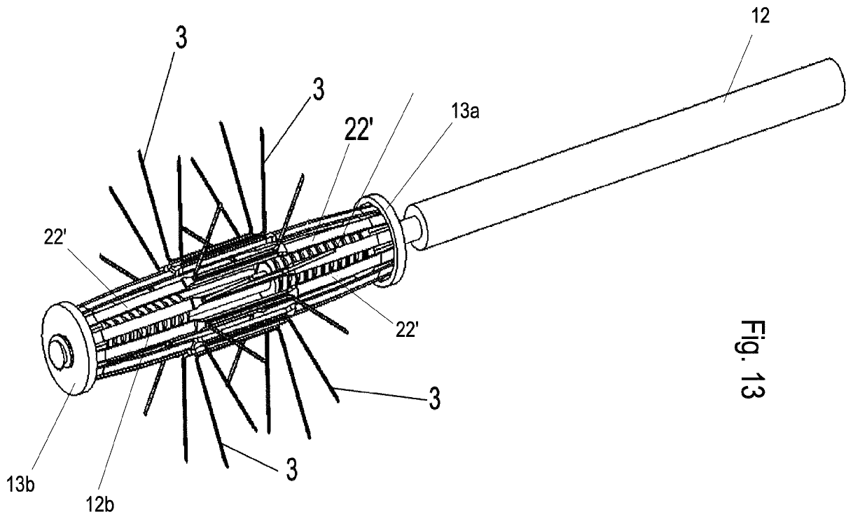

In FIG. 11, however, the supports 29 are shown in an unexpanded state. A push rod 6a is disposed in the housing 20, and a control plate 31 is disposed at the distal end of the push rod 6a, the control plate 31 having radially inwardly extending slots 31′ adjacent to a continuous edge 32 of the control plate 31 for the passage of radially inner portions of the supports 29. When the control plate 31 is pulled in the direction of the arrow 35 by means of the push rod 6a, the continuous edge 32 of the control plate 31 runs up on the link guide 30 in the supports 29 and the supports 29 are displaced or driven in the direction of the arrows 33 in accordance with the course of the link guide 30, as shown in FIG. 12. As a result, a primary implant arranged on the supports 29 is expanded or driven outward and can come into contact with a vessel wall. Once this is done, by actuating a mechanism not shown in detail to drive the penetrating members 3 outward, the clamp-like penetrating members 3 can be driven outward through the primary implant 2 and the vessel wall and, if necessary, through a secondary implant 17 and brought into contact with the restraining member 4, where they can be bent over.

In FIGS. 11 and 12, it can be seen that a penetrating member 3 is formed similar to a staple with a transverse connection between the tips facing outward. A penetration element 3 is received in the laterally open guide elements 22 in the embodiments according to FIGS. 7 to 12, so that a penetrating member 3 can slide outwardly along the respective guides or guide elements 22. This movement is caused by mandrils 22 guided in the portions of the guide elements 22 extending along the axis 34, which are deflected outwardly in the guide elements 22 and cooperate in contact with the penetrating members 3.

In FIG. 12, it can be seen that the supports 29 have recesses 36 to retain a primary implant, for example in the form of a tube of textile material, so that it is not released from the recesses 36 until it has been secured to the vessel wall by the penetrating members 3 when the supports 29 are retracted into the housing 20. Likewise, recesses 37 for a secondary implant may be provided on the restraining member 4. Guide curves in the restraining member 4 are denoted by 38, which ensure that the penetrating members 3 are bent over and thereby secured outside the vessel wall and, if necessary, a secondary implant when they run onto the restraining member 4 in the sense of the arrows 39.

In FIG. 13 it can be seen that guide elements 22′ for the penetrating members 3 are arranged in the housing 20, which represents a base body in the sense of the present invention, wherein, as already described above with another variant of the device 1 according to the invention, a guide rod 12 with two threaded sections 12a and 12b with threads running in opposite directions is mounted in the base body or housing 20, a screw 13a and 13b, which are mounted in a rotationally fixed manner relative to the base body, being arranged on each of the threaded sections 12a and 12b, and the flexible guide elements 22′ being arranged on the screws 13a and 13b and connecting the screws 13a and 13b. Thus, again on a single guide or threaded rod 12, two counter-rotating spindle drives are formed to move the screws 13a and 13b of the spindle relative to each other so that, as a result, the guide elements 22′ are bent as the screws 13a and 13b converge on the threaded rod 12 and are thereby driven radially outward. In this way, the penetrating members 3 are also driven outwards, as can be seen in FIG. 14.

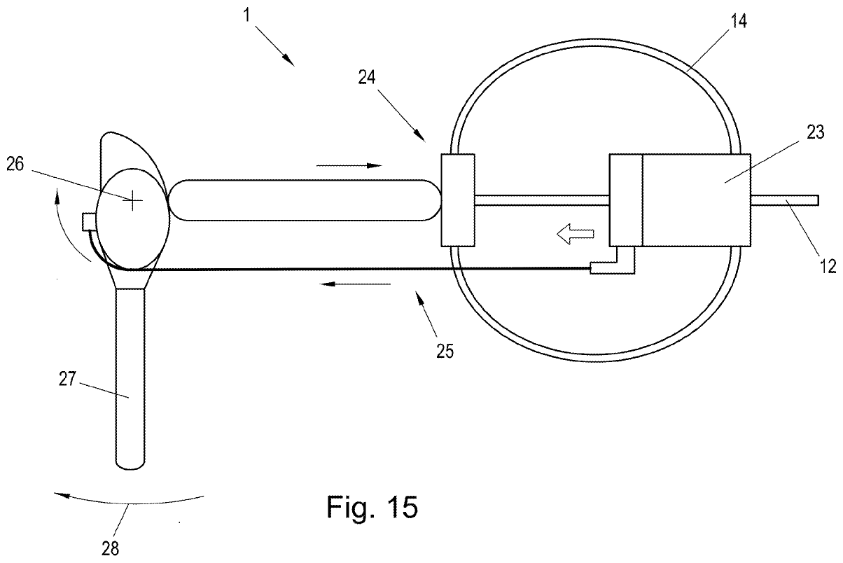

FIG. 15 shows that the carrier member has a guide rod 12 for a first bearing 23 for expansion elements 14, which is guided displaceably along the guide rod 12 and can be displaced, preferably by means of a cable pull 25, against a second bearing 24 for expansion elements 14, which is not displaceably mounted on the guide rod 12, the expansion elements 14 being fixed as a support structure to the bearings 23 and 24 and connecting the bearings 23 and 24. By actuating the handle 27 in the direction of the arrow 28, the cable 25 is rolled up on the pulley 26, which is preferably designed as an eccentric pulley, whereby the first bearing 23, which is slidably guided on the guide rod 12, is moved by the cable 25 along the guide rod 12 in the direction of the second bearing 24, which is not slidably guided. This expands or drives the expansion elements 14 outward.

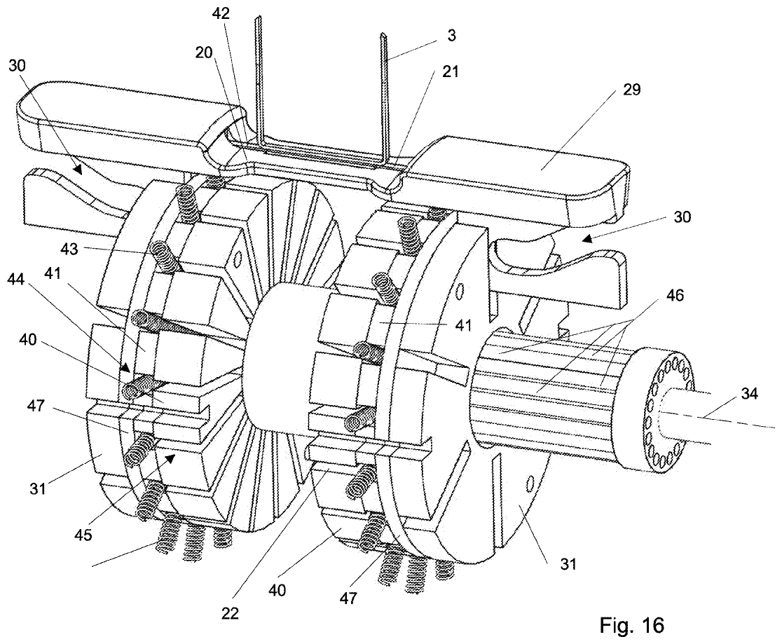

In FIG. 16 and the following figures, identical or corresponding parts are again marked with the same reference signs as in the preceding figures and in particular in FIGS. 7 to 12.

In FIG. 16, the housing 20 is only partially shown for clarity. However, it can be seen that a penetrating member 3 emerges from a slot 21 in the housing. The housing 20 also forms the supports 29 in the embodiment of FIG. 16. The guides 22 for the penetrating members 3 are formed in first disks 40 and second disks 41, which are coaxial with respect to the longitudinal axis 34 of the carrier member and spaced apart from each other. The guides 22 are formed in the disks 40 and 41 as recesses and slots, respectively, which extend radially outward. In the guides, the penetrating members 3 rest on internal supports 42 for the penetrating members 3, on which the penetrating members 3 can be driven outward by the action of the spiral springs 43. The spiral springs 43 are accommodated in second guide regions 44 of the guides 22, these second guide regions 44 being widened in the circumferential direction of the second disks 41 compared to the first guide regions 45 provided with the reference sign 45. In this way, the second guide regions 44 provide sufficient space for the spiral springs, while the guidance of the penetrating members 3 or the supports 42 takes place in the narrower first guide regions 44. Guide rods for the spiral springs 43 are designated by reference sign 46, the function of which will be explained later.

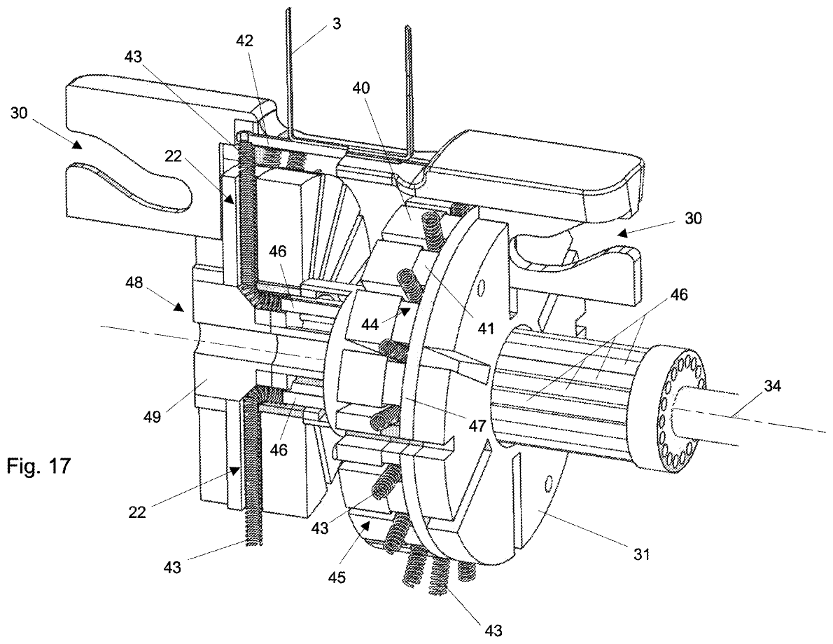

In FIG. 17, it can be seen that the disks 40 and 41, as well as the control plate 31 and a limiting plate 47, are mounted on a common, multi-piece shaft 48 that extends along the longitudinal axis 34. In the condition shown in FIG. 17, the spiral springs 43 are pushed by the guide rods 46 and extend lengthwise into the second guide regions 44 so that the supports 42 for the penetrating members 3 are driven outward. Here, the spiral springs 43 are guided by a cone 49, which forms part of the shaft 48, into the second guide regions 44 of the second disks 45.

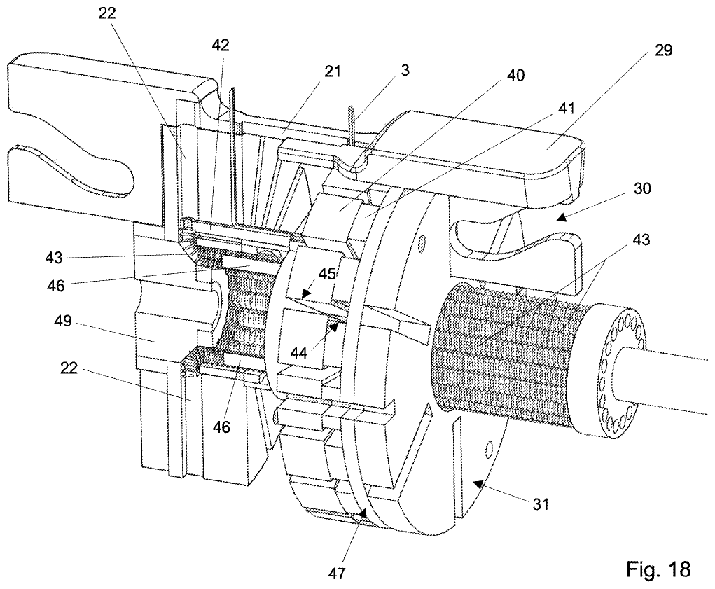

In FIG. 18, the spiral springs 43 are still slid onto the guide rods 46 and are just level around the bend in the area of the cone 49. It can be seen that in this condition the spiral springs 43 of the right disk group protrude from the disk group. Also, it can be clearly seen in FIG. 18 that the second guide regions 44 are widened in the circumferential direction of the second disk 41 to provide space for the thickened guide ends of the supports 42 and the spiral springs 43.

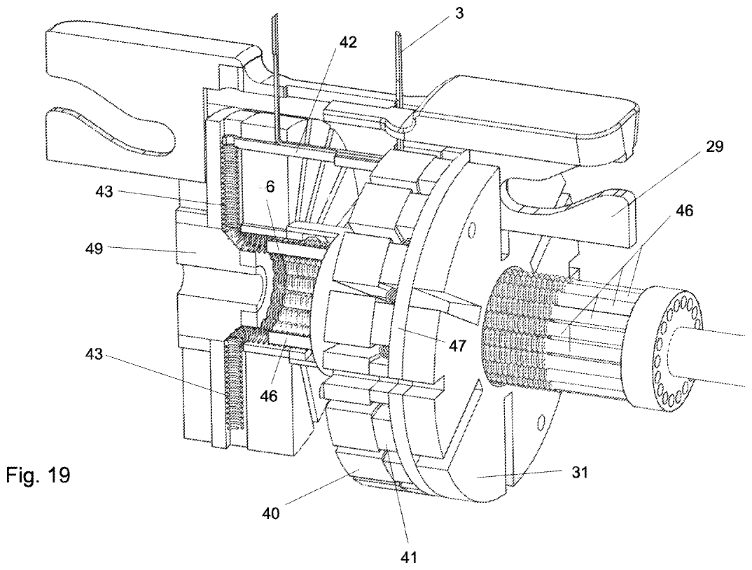

In FIG. 19, the support 42 is approximately halfway outward in the second guide region 44, and the support 42 thereby drives the penetrating member 3 outward in front of it.

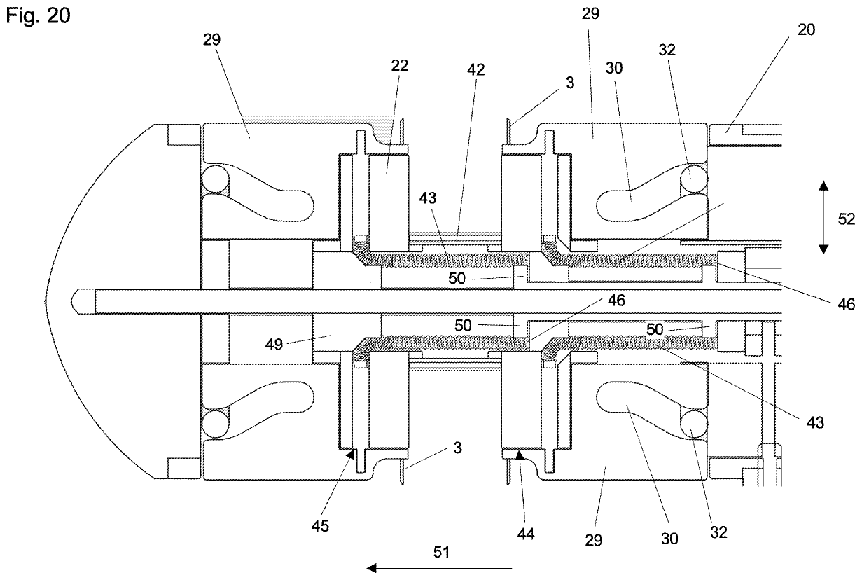

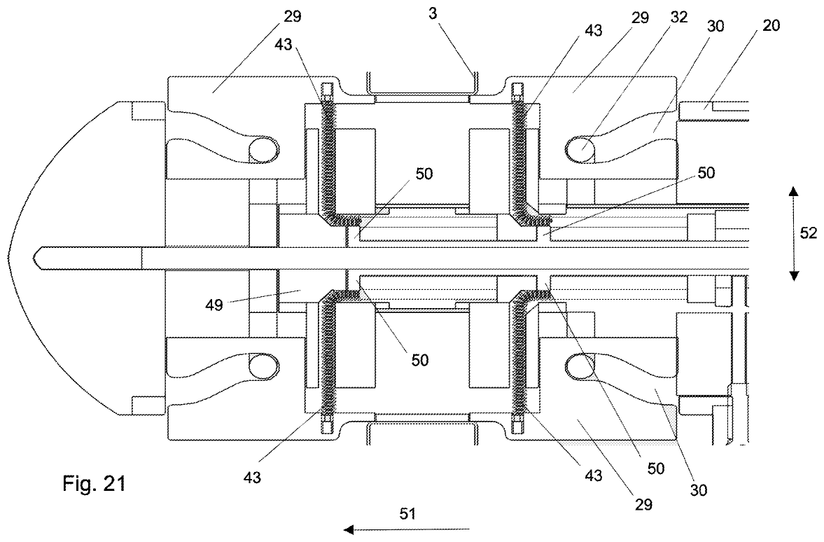

FIG. 20 shows the gripping elements 50 which, when actuated in the direction of arrow 51, push the spiral springs 43 off the guide rods 46. This results in a longitudinal expansion of the spiral springs 43, which were previously held compressed on the threaded rods 46, so that the spiral springs 43 enter the second guide regions 44 and drive the supports 42 or the penetrating members 3 outward. This is shown in FIG. 21, in which the gripping elements 50 are in the outwardly pushed position. The action of the link guide 30 together with the continuous edge 32 of the control plate 31 allows the supports 29 to be pushed outward in the direction of the double arrow 52. This can be done independently of the actuation of the gripping elements 50, for example, to support the vessel wall and the implant prior to the placement of the penetrating members 3, or coupled and thus simultaneously to provide a fixation site as quickly as possible.

1-33. (canceled)

34. A device for attaching a primary implant as a fixation site for a vascular prosthesis to a vessel wall of a blood vessel, the device comprising:
a carrier member configured to drive a plurality of penetrating members for penetrating the vessel wall radially outwardly with respect to a longitudinal axis of the carrier member;
wherein a restraining member is formed on the carrier member as a radial stop for the penetrating members.

35. The device according to claim 34, wherein the restraining member has an adjustable diameter.

36. The device according to claim 34, wherein the restraining member is formed as a ring surrounding the carrier member.

37. The device according to claim 34, wherein the restraining member is formed by a plurality of plates fixed to the carrier member.

38. The device according to claim 37, wherein the plurality of plates are fixed to the carrier member at a distance from the longitudinal axis adjustable in a radial direction.

39. The device according to claim 34, wherein the primary implant is arranged radially outside the carrier member and is detachably fixed to the device, the primary implant being penetrable by the penetrating members and being radially expandable.

40. The device according to claim 34, wherein the primary implant comprises a tube made of textile material.

41. The device according to claim 34, wherein the primary implant comprises a metallic tube having lattice walls.

42. The device according to claim 34, wherein the carrier member comprises a radially expandable common support structure for the plurality of penetrating members.

43. The device according to claim 34, wherein the carrier member has a housing and, in the housing, radially outwardly directed guides for the penetrating members, along which the penetrating members can be driven radially outwardly.

44. The device according to claim 43, wherein the carrier member comprises a radially expandable common support structure for the plurality of penetrating members, the carrier member comprising a central push rod which cooperates with a plurality of radially displaceable supports for the primary implant, the supports being drivable radially outwardly by an axial displacement of the push rod relative to the supports.

45. The device according to claim 43, wherein the radially outwardly directed guides for the penetrating members are formed in at least two first disks which are arranged coaxially and spaced apart from each other with respect to the longitudinal axis of the carrier member and comprise the guides as radially extending recesses.

46. The device according to claim 45, wherein the penetrating members can be driven radially outward in the guides on supports for the penetrating members.

47. The device according to claim 45, wherein the guides each have a second guide region which is widened in the circumferential direction of the disk compared to a first guide region adjoining the second guide region in the axial direction.

48. The device according to claim 47, wherein the second guide regions are formed in respective second disks adjacent to the first disks, the second disks having recesses extending radially to form the second guide regions.

49. The device according to claim 45, wherein the at least two first disks are mounted on a common shaft which has axial recesses or bores and guide rods received therein and spiral springs provided on the guide rods, the spiral springs each being at least partially withdrawable from the guide rods and insertable into a guide by a gripping element.

50. The device according to claim 49, wherein the gripping elements of all spiral springs are actuable together.
